(19) United States
(12) Patent Application Publication
(10) Pub. No.: US 2002/0192657 A1
(43) Pub. Date: Dec. 19, 2002

(54) Bio-tolerant substrata having analyte binding microarray

(76) Inventors: Robert L. Erwin, (US); N. Leigh Anderson, (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 09/883,358

(22) Filed: Jun. 19, 2001

(51) Int. Cl. C12Q 1/68; G01N 33/53; G01N 33/542; C12M 1/34; G01N 33/543

(52) U.S. Cl. 435/6; 435/7.9; 435/287.2

(57) ABSTRACT

Contact lenses or similar ocular devices are disclosed that have specific binding molecule microarrays printed on or embedded in them to bind various analytes present in tears. Tear are used as a non-invasive alternative to the monitoring of proteins and other constituents found in serum, especially low molecular weight analytes and low abundance proteins. The devices can be placed in a developing reagent or the subject wearing the device can perceive changes in the visual filed that can be used to diagnose disease status as well as monitor various physiological and ambient environmental (exogenous) conditions.

### FIELD OF THE INVENTION

The instant invention relates to contact lens and other devices that remain in extended contact with various biological fluids, where such devices contain microarrays of specific binding molecules. Uses and methods for manufacture of such devices are disclosed. The devices are constructed by depositing specific binding molecules on a bio-tolerant substratum or matrix at addressable locations. The devices are positioned to contact target body fluids, wherein after sufficient time, specific binding to selected components is determined.

### BACKGROUND OF THE INVENTION

There has been a growing interest in recent years in the non-intrusive clinical sampling of body fluids for detecting various substances. This is largely the result of improved analytical techniques and the realization that many of the components of physiological interest (e.g., toxins, metabolites, drugs, proteins, nucleic acids, infectious agents, allergens, etc.) contained in blood samples obtained by intrusive means are also contained in other body fluids such as urine, sweat, tears, sputum and saliva, which can be obtained much more easily and at reduced risk. In addition, these samples may be advantageous for testing for components of physiological interest (U.S. Pat. No. 4,635,488).

Tears are an excellent alternative biological source for physiologically relevant constituents. For example, immunoassays on tear proteins have been used to diagnose diseases (Inada et al., Jap J Opthalmol (1985) 29(2):212-21 and Mii et al., Electrophoresis (1992) 13(6):379-382) and to detect markers for hormones (Ranganathan et al., Biochem Biophys Res Comm (1995) 208:412-7), including those associated with inflammation (Richard et al., CLAO J (1992) 18(3):143-7). Further, immunoassay for IgE in tears and its correlation to serum IgE is known (Somos et al., Allergy Asthma Proc (2001) 22(2):81-6, Baudouin et al., Graefes Arch Clin Exp Opthalmol (2000) 238(11):900-4).

Tears are produced, in large part, due to the activity of the lachrymal gland. The primary lachrymal gland proteins, lactoferrin, lysozyme, and tear specific prealbumin combined with IgA and s-IgA, make up 93% of total tear proteins. However, other constituent proteins can include peroxidase, interferons (e.g., INF-γ), interleukins (e.g., IL-2, 11-4, and IL-5), melatonin and growth factors (e.g., EGF, TGF-α). Viruses, such as Herpes Simplex have also been detected (Pramod et al., Can J Ophthalmol (2000) 35(3):134-40). It has also been suggested that the constituents found in tears are representative of those found in the blood supply to the brain, because the palpebral conjunctiva is supplied by the ophthalmic artery, a branch of the internal carotid artery, a major supplier of the brain (U.S. Pat. No. 5,352,411).

Although tears are an alternative body fluid that can be analyzed, it is generally difficult or impossible to obtain a large enough tear sample to allow measurement or detection of constituents. To obtain such a volume of tears for research or analysis, investigators have generally been required to use artificial stimulation of tear production, for example, with tear-inducing chemicals, fans, and the like. The concentration of some solutes in tears is flow-dependent and therefore depends on the method of collection of the tears.

Analysis of a complex biological fluid such as tears presents other challenges in terms of sensitivity, resolution/discrimination and avoidance of analyte loss during sample preparation (www.kratos.com/Mapps/NewApps/Apps23.html). Further, the low volume of tear fluid that is readily obtainable without induction, combined with the desire to detect low abundance proteins and other dilute markers in tears, make traditional isolation and detection means impractical for reliable clinical diagnostic purposes. What is required is a reliable method that effectively concentrates tear components prior to analysis. Further, such a method should allow for selective sequestration of said components, including ease of detection once selected.

In Erwin, U.S. Pat., No. 4,946,450, applicants have previously patented an ocular device for slow release of a compound to the eye for therapeutic purposes.

In Anderson et al, WO 01/09607, applicants have previously patented microarays and various methods for preparing microarrays.

In Braatz et al, U.S. Pat. Nos. 4,886,866 and 5,034,458, polyurethane based hydrogel extended wear contact lenses are disclosed.

### SUMMARY OF THE INVENTION

One means to achieve such a method, which is described in the present invention, is to generate and use modified substrata or matrices (e.g., contact lenses) as “continuous collectors.” By placing a modified substratum, which selectively segregates tear components, in the eyes for different periods, a large volume of tears can pass over and through the substratum, thereby concentrating desired components without the limitations that would be expected from low tear volume. Further, the selective aspect of the modified substratum can be achieved by generation of a microarray surface comprising discrete analyte-assay regions on the substratum, where each discrete region in the microarray has a selected, analyte-specific reagent (e.g., antibody).

The method and device of the present invention allows determination of various biological constituents present in tears such as proteins, nucleic acids, peptides, viruses and bacterial components as well as organic tear constituents such as glucose in an efficient and non-invasive manner.

In one aspect, the inventions relates to an ocular device comprising a selective one or two-dimensional array having a plurality of specific binding molecules, where said device selectively binds moieties comprised in ocular secretions upon contacting an ocular surface with said device.

In a related aspect the specific binding molecules can include, but are not limited to, antibodies (including label conjugated antigen bound to antibodies), nucleic acids, proteins, receptors and other relatively specifically binding or reacting molecules or structures which are deposited at addressable locations on the device. In a further related aspect, such deposited materials can be adsorbed to the surface or internal structures or bonded to the surface or internal structures of the device by covalent or non-covalent means. The device may comprise opposing substantially convex and substantially concave surfaces, at least one surface of which comprises the deposited specifically binding molecules.

In a further aspect of the invention, a contact lens may be ground from a block of contact lens material that already contains microarray components already embedded in the block such that the microarray components are present before and after grinding the contact lens.

In another aspect the device may be, but is not limited to, contact lenses for in situ diagnostic use. For such a use, the contact lenses can be produced for extended wear purposes, and thus synthesized from extended wear materials. The contact lens may have vision correcting ability or may not affect the focal length at all.

In another aspect, the device may be, but is not limited to, non-ocular prosthetic devices to be implanted for in vivo corrective use or other non-ocular devices to contact body fluids. Examples include catheters, dialysis equipment, shunts, insulin infusion devices, blood bags, plasmaphoresis, inplantable objects/devices, including partially implanted objects such as sutures, surgical staples etc., which contact body fluids for a period of time. While performing their otherwise intended function, such devices simultaneously have an array of specific binding agents which are performing a diagnostic function. This is particularly significant for therapeutic inplantable devices which have a therapeutic function such as a prosthesis or valve or even staples and sutures. The diagnostic portion of the device should contact various body fluids while not interfering with the basic function of the inplantable device. Likewise, a vessel or conduit for holding body fluids or circulating body fluids may have the array present on the inside of the vessel or conduit in contact with body fluids.

Another aspect is to provide a simple and reliable diagnostic screening system for noninvasive evaluation of ocular secretions comprising contacting an ocular device comprising a selective array with the ocular surface of a subject. In a related aspect, the array is allowed to interact with the surface for a specified period of time, the device is then removed and subsequently, the presence, absence or amount of an analyte contained in said secretion that selectively binds to the device is determined. Further, such a determination can be correlated with a specific diagnosis.

In a further related aspect, the device may be separated from bound analytes and determination of the presence of an analyte can be carried out any conventional means such as a specific binding assay, for example an immunoassay.

Further, such diagnosis can be used to determine disease markers including those associated with long term conditions or hard to detect early conditions such as silent heart attacks, silent stroke, tumors and diabetes. In addition, the specific binding molecules can detect or interact with any metabolite, protein, nucleic acid, or organic molecule, including but not limited to, hormones, cytokines, lymphokines, interleukins, interferons, chemokines, and tumor, viral, bacterial, and T-cell, antigens and nucleic acids.

In another related method, diagnosis using ocular secretions in the absence of the device removal is disclosed, whereby analyte-specific binding molecule interaction is physically perceived by the subject by, for example, a perceptible change in color or production of a mark or symbol visualized by the subject. Such perceptible reaction can be in response to the presence of an endogenous analyte (e.g., luteinzing hormone, response to a vaccine by production of specific immunoglobulins against vaccine epitopes etc.) or an exogenous analyte, particularly a toxic chemical.

In a related aspect, a method for determining a toxic or effective response to a drug is disclosed comprising contacting the device comprising a one or two dimensional array having a plurality of specific binding molecules with a body fluid of a dosed subject, where determining the presence of a toxic or effectiveness breakdown substance(s) or products or changes in the abundance of proteins or nucleic acids altered thereby which correlates with a negative or positive response to a drug. In another related aspect, a method of determining the response to an allergen is disclosed comprising determining the presence of, for example, IgE using such a device.

### BRIEF DESCRIPTION OF THE DRAWINGS

FIG. 1 depicts a contact lens with a microarray of specific binding molecules bound thereto in addressable locations.

FIG. 2 is a sectional view of a block of contact lens material to be ground containing embedded microarray material.

FIG. 3 depicts tubing for dialysis with a microarray of specific binding molecules bound to the inside surface thereof in addressable locations.

FIG. 4 is a sectional view of a mold with a preformed microarray placed therein before adding the contact lens material.

### DETAILED DESCRIPTION OF THE INVENTION

The terms “specific binding molecule”, “molecule of interest”, “agent of interest”, “ligand” or “receptor” may be any of a large number of different molecules, complexes, biological cells or aggregates, and the terms are used interchangeably. Each specific binding molecule is immobilized at a cell, sector, site or element of the array and binds to an analyte being detected. Therefore, the location of an element or cell containing a particular specific binding molecule determines what analyte will be bound. Proteins, polypeptides, peptides, nucleic acids (nucleotides, oligonucleotides and polynucleotides), antibodies, ligands, saccharides, polysaccharides, microorganisms such as bacteria, fungi and viruses, receptors, antibiotics, test compounds (particularly those produced by combinatorial chemistry or known binding agents), plant and animal cells, organelles or fractions of each and other biological entities may each be a binding component if immobilized on or in the device. Each, in turn, also may be considered as analytes if it binds to a binding component on the device. The specificity of the binding may not be absolutely unique in all situations; for example, a lectin may bind more than one analyte.

The term “ocular device” means, for example, a contact lens, eye shield or other device for contacting the tears regardless of whether it actually corrects vision or not. In a related aspect, “contact lens” means a curved shell of glass or polymer applied directly over the globe or cornea. Such ocular devices are to be designed for use as diagnostic means in the present invention but may involve visual correction (i.e., to correct refractive errors).

The term “microarray” means a discrete analyte-assay region on a solid support, where each discrete region in the support has a selected, analyte-specific reagent.

When a specific binding molecule has a high molecular weight, it is referred to as a “macromolecule”. In terms of some biopolymers, the high molecular weight refers to greater than 100 amino acids, nucleotides or sugar units long.

The term “bind” includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measured. The “binding” interaction is usually semipermanent but may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

In a preferred embodiment, the present invention relates to methods of designing and producing a member of a binding pair that specifically binds to its partner as well as to the products resulting from these methods. Such members are referred to herein as specific binding molecules.

In a related aspect, “specific binding molecule” also refers to an entity, e.g., a molecule, complex or a portion thereof, which binds to a target.

The term “target” means an entity with which a specific binding molecule binds. Methods of the invention optimize binding affinity between a target and a specific binding molecule. A target can be a molecule, a portion of a molecule, or an aggregate of molecules. A target and a specific binding molecule can be separate molecules, or they may be different moieties on one molecule. A target includes a target site. A target may be capable of reversible attachment to a binding molecule via a tether. Examples of targets include: nucleic acids (e.g., RNA or DNA, double stranded DNA, single stranded DNA, or supercoiled DNA), peptides or proteins (e.g., enzymes, receptors or antibodies), carbohydrates, and other molecular structures, such as nucleic acid-protein complexes, chromatin or ribosomes, lipid-bilayer containing structures, such as membranes, or structures derived from membranes, such as vesicles.

The term “matrix” means ground substance in which things are embedded. In a related aspect, the term “substratum” means a solid surface or support. The two terms may be used interchangeably.

The term “bio-tolerant” means that a material does not cause an allergic, toxic, pyrogenic or other noxious reaction when placed in contact with a biological surface and is generally well tolerated by the host organism.

The term “tether” means a structure that includes a moiety capable of forming a reversible or non-reversible bond with another moiety (e.g., a moiety on another tether) and (optionally) a spacer element. Alkane chains are suitable spacer moieties.

The term “hydrogel” means a material, which absorbs water, in the range of 10 to 95 percent by weight, without itself dissolving.

The term “analyte” means a microorganism, molecule, typically a macromolecule, such as a polynucleotide or polypeptide, or complex whose presence, amount, and/or identities are to be determined. The analyte is one member of a ligand/anti-ligand pair. For example, such a ligand anti-ligand pair would exist when an anti-idiotypic antibody is directed against a receptor binding domain. Such an anti-idiotypic antibody could then bind to a ligand of said receptor.

The term “two dimensional array” means a linear arrangement of regions on a solid support of preferably discrete regions, each having a finite area, formed on the surface of a solid support.

The term “dosed” means a drug has been administered to a subject.

The term “addressable location” means a region that can be identified by an address or a name for information transfer.

The terms “substantially convex” and “substantially concave” refer to the curvature that the ocular device might have; i.e., curving outward or inward, respectively.

The term “surrounding ambient environment” means the external atmosphere or milieu to which the subject wearing an ocular device is exposed.

The term “extended wear” means a material or surface of a material which may be in intimate contact with the eye for an extended period of time without significantly damaging the eye and without excessive user discomfort. For example, in one embodiment, the period of extended continuous contact is at least about 24 hours. In a related embodiment, the period of extended continuous contact is at least about 4, 7 or 14 days. In a preferred embodiment, the period of extended continuous contact is at least about 30 days.

The term “ocular surface” relates to any available plane of the eye. In a preferable embodiment, the ocular surface is the external or superficial aspect of the eye. In a more preferable embodiment, the surface is of a human eye.

The term “ocular secretion” means the watery secretion of the lachrymal glands, which serve to moisten the conjunctiva, including what is commonly called tears.

The term “allergen” means an antigenic substance capable of producing, for example, but not limited to, an immediate type hypersensitivity reaction (allergy).

### I. CONTACT LENS MICROARRAYS

Ocular Devices

In order to produce the devices of the present invention, materials with very high water content are desired, e.g., hydrogels. Production of such materials for contact lenses is well known in the art. For example, see U.S. Pat. Nos. 6,164,777; 5,610,204; 5,451,617; 5,449,729; 5,387,632; 5,352,714; 5,252,628; 5,158,717; 4,981,487; 4,946,450; 4,865,439; 4,578,230; 4,564,484; and 4,218,554. In a preferred embodiment, it has been desired to provide hydrophilic polymers and contact lenses made therefrom which are of high water content, high strength, good durability, high gas permeability and which can be repeatedly disinfected or sterilized by thermal means without deterioration or destruction or impairment of their optical properties.

In another preferred embodiment, the ophthalmic compatibility requirement for the ocular devices of the present invention is that the devices must allow oxygen to reach the cornea in an amount that is sufficient for long-term corneal health. The contact lens must allow oxygen from the surrounding air to reach the cornea because the cornea does not receive oxygen from the blood supply like other tissue. If sufficient oxygen does not reach the cornea, corneal swelling occurs. Extended periods of oxygen deprivation cause the undesirable growth of blood vessels in the cornea. “Soft” contact lenses conform closely to the shape of the eye, so oxygen cannot easily circumvent the lens. Thus, in a preferred embodiment, ocular devices comprise soft contact lenses that allow oxygen to diffuse through the lens to reach the cornea. In a related aspect, the ocular device of the present invention should be manufactured and comprise materials substantially as those comprising “extended wear” lenses (see for example, U.S. Pat. Nos. 5,965,631; 5,849,811; 5,789,461; 5,776,999; 5,760,100; 5,084,537; 5,002,979).

In a related aspect, the device must not strongly adhere to the eye. In the most common embodiment, the subject must be able to easily remove the lens from the eye for analysis when performing home diagnostic tests. However, the device must also be able to move on the eye in order to encourage tear flow between the device and the eye such that the collection function of the device can be accomplished. Tear flow between the device and eye allows for percolation of the fluid through the plurality of specific binding molecules. Thus, the device must not adhere to the eye so strongly that adequate movement of the lens on the eye is inhibited.

While there exist rigid gas permeable (“RGP”) contact lenses which have high oxygen permeability and which move on the eye, and may be used for the present invention, RGP lenses are typically quite uncomfortable for the subject. Thus, soft contact lenses are the preferable material to use for the ocular devices because of comfort. Moreover, a device that may be continuously worn for a period of a day or more (including wear during periods of sleeping) requires comfort levels that may exclude RGP lenses in certain applications.

In a preferred embodiment, contact lenses or other ocular devices will have an antibody microarray printed on (or embedded within them) to bind various proteins and other analytes in tears over a period of time. In a related aspect, the devices can be removed and immunoassays performed thereon. In another aspect, the devices can remain on the subject and the subject can perceive a color change on the device. Such a device can be used, for example, to detect toxicity of systemic chemotherapy of anti-neoplastic drugs through analysis of tears (Cruciani et al., Ann Opthalmol (1994) 26(3):97-100) or the presence of anti-oxidant enzymes (Crouch et al., Ocul Pharmacol (1991) 7(3):253-258). In a related aspect, specific antibodies and chemokines (e.g., including, but not limited to, interleukins, interferons, cytokines) may be identified which are associated with specific syndromes such as auto-immune and inflammatory disorders (e.g., but not limited to, lupus, ARDS, arthritis, psoriasis etc.). Further, in a preferred embodiment, pollutants can be measured, such as exposure to ozone (Schmut et al., Free Radic Biol Med (1994) 17(2):165-169) or Red Child Syndrome (Salazar de Sousa et al., Arch Dis Child (1987) 62(11):1181) may be diagnosed using such a device. While such a device may be used to monitor disease and various conditions both external (exogenous) and internal (endogenous), the devices may also be used for correction of visual impairment simultaneously (i.e., for correction of refractive errors).

In another aspect, the array comprises orientation markers. This permits one to orient the microarray on the device. One may also have a one-dimensional array be in the form of a line of spots such that when the device is spun, the spots form a type of bar code, which may easily be detected.

It is still another aspect of the invention to have positive and negative control regions on the microarray as a quality control check for the assay.

It is another aspect of this invention to provide a method and device that maintains a constant flow of tears. It is a further aspect of this invention to provide a method and device having embodiments, which may be graded either semiquantitatively or quantitatively. It is a further aspect of the invention to provide a non-invasive method and device that may be used in the home or other non-clinical settings for the determination of organic and ionic constituents in tears.

In a preferred embodiment, the device may be used for a short period of time. For example, the device may be used in an ophthalmologist's office to diagnose a bacterial/viral infection, eye damage and other eye disease (e.g., general means of monitoring lachrymal gland secretions for antibodies, changes in tear film components [e.g., growth factors and corneal wound healing, etc.]). In a related aspect, the device may be used for a short period of time (e.g., about 5-30 minutes) or for longer periods (e.g., up to and maybe more than 30 days).

The advantage of the present invention is the portability of the device, especially for outsourced analysis (e.g., physician or patient can mail the device to a laboratory). This device will also allow for reduced handling and degradation that is inherent in urine and blood sample analysis.

### Microarrays

A microarray is essentially a two-dimensional support or sheet wherein different portions or cells (sectors) of the support or sheet carry different biomolecules or elements, such as, oligonucleotides, nucleic acids, peptides, polypeptides, saccharides or polysaccharides, bound thereto. Microarrays are similar in principle to other solid phase arrays except that assays involving such microarrays are performed on a smaller scale, thus allowing reduced reagent usage and lower sample consumption. Microarrays have been used for a number of analytical purposes, typically in the biological sciences.

Biochemical molecules on microarrays have been synthesized directly at or on a particular cell (sector) on the microarray, or preformed molecules have been attached to particular cells (sectors) of the microarray by chemical coupling, adsorption or other means. The number of different cells (sectors) and therefore the number of different biochemical molecules being tested simultaneously on one or more microarrays can range into the thousands. Commercial microarray plate readers typically measure fluorescence in each cell (sector) and can provide data on thousands of reactions simultaneously thereby saving time and labor. A representative example of a patent in the field is U.S. Pat. No. 5,545,531.

Currently, one and two dimensional arrays of macromolecules are made either by depositing small aliquots on flat surfaces under conditions which allow the macromolecules to bind or be bound to the surface, or the macromolecules may by synthesized on the surface using light-activated or other synthetic reactions. Previous methods also include using printing techniques to produce such arrays.

Some methods for producing arrays have been described in “Gene-Expression Micro-Arrays: A New Tool for Genomics”, Shalon, D., in Functional Genomics; Drug Discovery from Gene to Screen, IBC Library Series, Gilbert, S. R. & Savage, L. M., eds., International Business Communications, Inc., Southboro, Mass., 1997, pp 2.3.1.-2.3.8; “DNA Probe Arrays: Accessing Genetic Diversity”, Lipshutz, R. J., in Gilbert, S. R. & Savage, L. M., supra, pp 2.4.1.-2.4.16; “Applications of High-Throughput Cloning of Secreted Proteins and High-Density Oligonucleotide Arrays to Functional Genomics”, Langer-Safer, P. R., in Gilbert, S. R. & Savage, L. M., supra; Jordan, B. R., “Large-scale expression measurement by hybridization methods: from high-densities to “DNA chips”, J. Biochem. (Tokyo) 124:251-8, 1998; Hacia, J. G., Brody, L. C. & Collins, F. S., “Applications of DNA chips for genomic analysis”, Mol. Psychiatry 3:483-92, 1998; and Southern, E. M., “DNA chips: Analyzing sequence by hybridization to oligonucleotides on a large scale”, Trends in Genetics 12:110-5, 1996. In a preferred embodiment, such microarrays are produced by depositing specific preformed binding molecules onto the surface of an ocular device. For example, the microarray is made by conventional dab printing technology (e.g., see U.S. Pat. Nos. 5,896,935 and 5,008,426) using antibodies. The receptors are deposited by dispensing small droplets, such as by inkjet printing, small pipette droplets, piezoelectric printing, and contacting pins (or other styles) dipped in receptor containing solution. Similar methods are now commercially used to print exotic patterns on contact lenses for decorative purposes. The receptors may bind to the surface or diffuse into and bind to internal structures of the contact lens. If the contact lens is formed by two layers, the printing may be performed between the two layers.

The microarray also can be constructed in contact lenses by applicant's methods in WO 01/09607 where the physical microarray may be bound to the surface, embedded in the contact before polymerization or added to the outside edge of the microarray as an outer rim.

Contact lenses are ground from glass or polymeric material or injection molded. A particularly preferred embodiment is where a portion of the contact lens material, preferably not the central portion for vision, may be made from fibers containing specific binding molecules which are bundled and embedded in the matrix of the contact lens material. This is the method of WO 01/09607 where large sections are taken and then ground to form contact lenses. Alternatively, the preformed microarray or fibers thereof may be added between layers of a contact lens or be placed inside the mold before polymerization of the contact lens material.

In addition to methods by which a receptor or molecule of interest is immobilized and used to bind an analyte, general methods exist for immobilizing members of a class of reactants. For example, protein A or protein G may be immobilized and used subsequently to bind specific immunoglobulins, which in turn will bind specific analytes. A more general approach is built around the strong and specific reaction between other ligands and receptors such as avidin and biotin. Avidin may be immobilized on a solid support or attached to a gel and used to bind antibodies or other reactants to which biotin has been linked covalently. That allows the production of surfaces to which a variety of reactants can be attached readily and quickly (see Savage et al., Avidin-Biotin Chemistry: A Handbook. Pierce Chemical Company, 1992). In a related aspect, protein antigens may be obtained from 2-D gels and used to produce antibodies for deposit on the device.

Alternatively, an embodiment may also comprise conventional contacts or specially treated contacts without antibodies, which passively absorb tear components (analytes). The contact is removed and adsorbed analytes are stripped off and separately measured. In another preferred embodiment, the microarray is positioned between lens material layers to produce an array within an ocular device.

A wide variety of methods has been developed to detect reactions between immobilized molecules of interest and soluble reactants. The methods differ chiefly in the mechanism employed to produce a signal and in the number of different reagents that must be sandwiched together directly or indirectly to produce that signal. These formats are well known per se and encompass several subclasses of patents. Examples include so-called sandwich assays; the result is the immobilization in the detection complex of an enzyme that, in combination with a soluble substrate, produces a preferably insoluble dye that may be fluorescent. Alternatively, the detection complex attached to the bound analyte may include a dendritic molecule, including branching DNA, to which is attached many fluorescent dye molecules. In a related aspect, there are fluorescent dyes that bind directly to agents of interest. For example, rare earth metal chelates can be used such as, but not limited to, holmium, europium, terbium, samarium, ytterbium, neodymium, and dysprosium. In a preferred embodiment, the rare earth metal is europium. In a further related aspect, heavy metals such as, but not limited to ruthenium can be used. These dyes are available commercially from, for example, Molecular Probes, Inc. (i.e., SYPRO® Ruby Protein gel stain [ruthenium] and SYPRO® Rose Protein blot stain [europium]).

Study of deposits directly on contact lenses is possible by the surface spectroscopic techniques of X-ray photoelectron spectroscopy (XPS). In a preferred embodiment, Matrix assisted laser desorption/ionization mass spectroscopy (MALDI) is adapted to a surface oriented role and used in the characterization of proteins adsorbed both in situ and in vivo onto contact lens surfaces (see e.g., www.kratos.com/Mapps/NewApps/Apps23.html).

### EMBODIMENTS

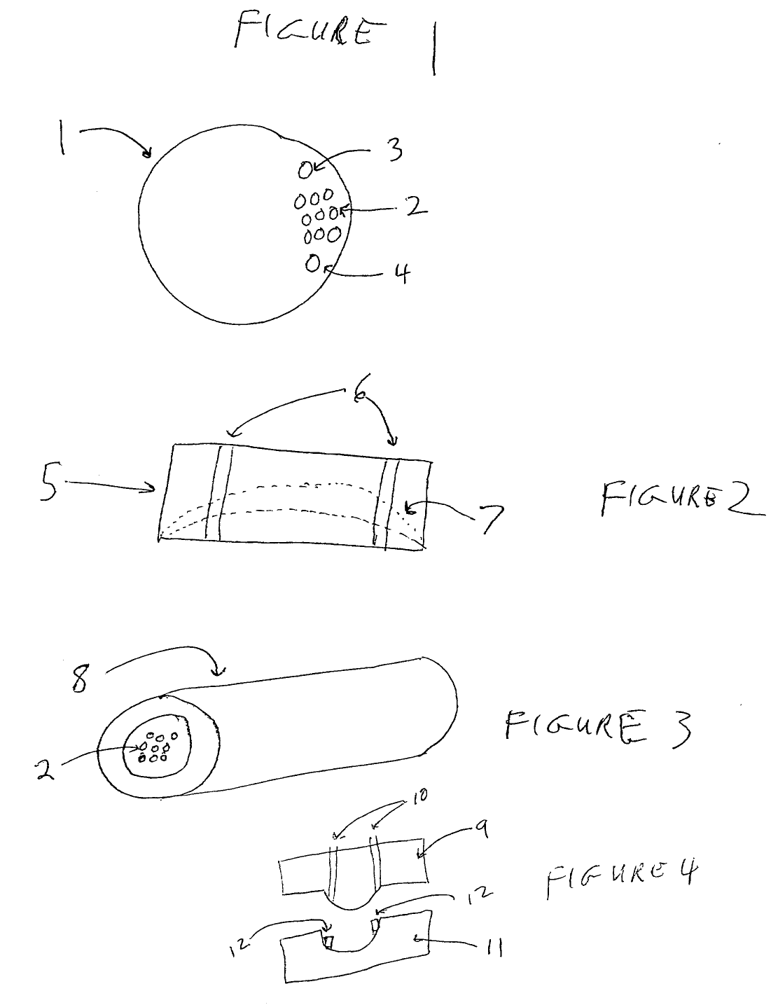

A frontal view of a contact lens (1) having a microarray (2) contained in or on it is shown in FIG. 1. Positive control (3) and negative control (4) provide an internal quality control check for the lens.

Before grinding or other cutting of a contact lens from a block of contact lens material (5), fibers of immobilized specific binding molecule (6) are embedded in block (5) as shown in FIG. 2. After forming the contact lens, the eventual shape indicated by dotted lines (7), the specific binding molecule will be present on only specific areas on the contact lens.

Other non-ocular devices that contact body fluids may be used such as tubing 8, which has the microarray (2) printed on the inside as shown in FIG. 3.

When contacts are prepared by injection molding, a preformed microarray or component (12) is preferably added to one half of the mold (11) before injection of contact lens material through ports (10) in the other half of the mold. Optically clear portions of the mold (not shown) may be present to permit photopolymerization of the contact lens material.

In a preferred embodiment, the contact lens material (e.g., HEMA, PVA, silicone, polyurethane, collagen, etc) can comprise surface chemistry to crosslink biochemical molecules (e.g., specific binding molecules) onto an ocular device (e.g., lens). This may be performed by plasma treatment to provide reactive chemical moieties (U.S. Pat. Nos. 6,224,948; 6,169,127; 5,962,136).

In another embodiment, the device is embedded with a protease inhibitor to prevent lysozyme, bacterial and other proteases from degrading the specific binding molecules present on the device.

In another embodiment, the device is removed from the subject's ocular surface and adsorbed proteins are removed or stripped (or the contact is dissolved; e.g., polysaccharide contact with amylase digestion), and subsequently the analyte is tested by conventional immunoassay. In a related aspect, the device serves as a continuous collector of tear components. For example, for those subjects having chronic diseases, or for continuous monitoring of the tear components to signal the occurrence of a silent heart attack or stroke.

In another embodiment, diabetics can use the devices of the present invention to monitor blood sugar levels. The glucose concentration of tears remains invariant until the blood sugar exceeds the threshold level. When the level of plasma glucose rises to 200 mg/dl or 10 mM it may be correlated with the hyperglycemic elevated levels, and tear glucose can be used as an index for blood glucose concentration. See, Van Harringen et al., Albret von Graefes Arch. Klin. Ophthalmol. 202:1, 1977). In a preferred embodiment, antibodies to glycosylated proteins (Gonzales et al., Rev Med Child (2001) 129(2):141-8) can be deposited onto the device and used to monitor the accumulation of glycosylated products, which correlates with long term excess plasma glucose levels. The method of the invention allows home monitoring of glucose in the tear fluid as a substitute for blood or urine glucose determination as a method of guiding insulin therapy in diabetes mellitus.

In another embodiment, the device may serve as a means to diagnose pregnancy (HCG measurement) or fertility (hormones) in the home or as an out patient test in a physician's office. In a related aspect, the contact may be left in place for days to weeks and liters of tears may pass over the device during that time such that rare analytes, which are undetectable in 20 ml of blood, can accumulate until they reach detectable levels.

In another embodiment, the device may be put in a small container with a detection system such as a labeled second antibody (optionally biotinylated) and a detection reagent system.

In a preferred embodiment, correct orientation of the microarray on the device may be accomplished by orientation markers on the periphery of the device or within the microarray pattern itself. For example, a bar code can be included on the matrix. In a related aspect, one may spin the contact to give a circular pattern, which may be read in a manner similar to a barcode.

In another embodiment, after printing the antibodies on the contact lens, an antigen conjugated to a label can be used. In a preferred embodiment, the conjugated label is an enzyme having chemiluminescent or color forming properties in a suitable detection reagent system. When the complex is bound, the enzyme is sterically blocked. Thus, for example, when the antigen or hapten in tears is contacted, it competes with the antigen-enzyme conjugate to release it. The released enzyme is then activated and may metabolize ATP or other substances in tears or embedded in the device to produce a color change that is noticeable by the patient. In a related aspect, the device is on the eye or the assay may be performed on a device once removed from the eye with addition of a detection reagent optional. In another embodiment, different regions of the device interact with the free enzyme to form a colored precipitate or fluid that is apparent on the device. The principle of such a design with competitive binding is well known for in-vitro assays, such as the EMIT system (SYVA Corp.) (see e.g., Randall et al., Ther Drug Monit (1981) 3(3):311-2).

In a related embodiment, the color may be apparent to the subject wearing the device. In a related aspect, if the antibody pattern is within the field of vision, the subject wearing the device may receive certain messages depending on the pattern, as different antibodies bound would result in distinct patterns (e.g., including, but not limited to, a black X or a color change in or outside the field of vision indicates the subject forgot to take the required dosage of medicament). Other labels such as colored materials, optical absorbers, optical reflectors, index of refraction changing materials; color changing with view angle, liquid crystals, etc may be used. The same labels may be used for non-competitive “sandwich type” assays also, Of particular interest is the use of pairs of fluorescent and quenching moieties (FRET sets). One of these is bound to the analyte and the other is bound to or near the specific binding molecule. When bound, little to no fluorescence is noticed. However, upon competitive binding with the analyte, either the device or the free liquid around it becomes fluorescent. Other detection labels and combinations thereof are known per se and may be used.

In another embodiment, the device may be used by the military or police as an early warning system for exposure to tear gas, mustard gas, nerve gas, toxins or biological weapons. In a related aspect, such a system may be used to detect air pollution, factory pollutants, noxious fumes, toxic waste or combustion sources (vehicles, open flames, boilers etc.) as the chemical equivalent of a dosimerty badge.

In one embodiment, response to vaccines can be monitored. If the patient mounts an immune response, the contact will have antibody bound to that portion of the device. In a related aspect, the bound vaccine can be detected with labeled protein A or G or anti-human Ig antibody, the label being denser at the site of complex formation. This is particularly useful for tumor vaccines in order to know options for further treatment. One embodiment of the present invention involves the use of Non-Hodgkin's Lymphoma (NHL) vaccine treatment where the specific B-cell antigen is used as an immunogen. Historically, patients who respond immunologically have very long term survival rates (essentially cured) whereas those patients who do not respond immunologically have essentially the same survival rate as untreated patients (Hsu et al, Blood 89(9):3129-35 (1997)). Such vaccines may be prepared and used by the methods of McCormick et al Proc. Natl. Acad. Sci. 96(2):703-8 (1999). This is particular important for patients who receive the vaccine before chemotherapy to determine prognosis and whether the patient need undergo chemotherapy.

Another use for the present invention is for organ transplant recipients. The monitoring of anti-rejection drugs, such as cyclosporin is critical as the toxic and effective amounts are very similar. Thus, constant monitoring is desirable of the drug and/or its metabolites and/or proteins, which are altered in abundance due to the drug(s).

In another embodiment, the device comprises antibody to proteins produced in the serum in response to a negative reaction to a drug to warn the patient of a possible toxic side effects to the administered compound or possible inappropriate interaction between incompatible drug combinations. Certain drugs (e.g. statins) do not cause immediate toxicity but in some individuals, long term toxicity is a problem. Other drugs (e.g. warfrin) require constant monitoring and adjustment. These individuals require constant monitoring, which may be effectively, performed using the present invention.

In another embodiment, allergens may be printed on the device. In such a system, IgE may be detected by adding labeled anti IgE antibody to determine allergic responses to a specific allergen.

The invention is further illustrated in the following non-limiting examples.

### EXAMPLE 1

### Preparation of Contact Lens Material

1 cm×1 cm×1 mm blocks of hydrated polyurea-polyurethane polymer gel, typically produced for extended wear contact lens material, are prepared by the method of U.S. Pat. No. 5,039,458. The block is partially dehydrated and immersed in 5 mg/ml N-succinimidyl 6-(4′-azido-2′-nitrophenylamino) hexanoate (SANAH) (Pierce) in DMF in the dark. The block is irradiated with a UV lamp (˜330 nm) focused on a single spot, less than 1 mm in diameter on the block, for 5 minutes. The block is then rinsed in saline solution in the dark and soaked in saline solution five times with the solution changed every hour. A solution of human serum albumin 10 mg/ml in 50 mM sodium bicargonate buffer, pH 8.5, is added to the block for 1 hour at room temperature. The block is rinsed with saline solution and then incubated for one hour at room temperature with 0.1M ethanolamine. The block is then rinsed with saline solution and then stored in saline at 4° C. until ready for use. 10 μg/ml FITC labeled anti-human serum albumin is added to the block and fluorescence is noted at one spot on the block.

### EXAMPLE 2

### Contact Lenses for Determining Responsiveness to Non-Hodgkin's Lymphoma Vaccines

The unique cryptic immunoglobulin gene produced in B-cell lymphomas from each patient was cloned and expressed as a single chain antibody and administered to patients as a personalized non-Hodgkin's lymphoma (NHL) vaccine as part of a phase I clinical trial.

The method of Example 1 is repeated with the extended wear contact lens of the same material and the individualized NHL vaccine. The protein spot is placed outside the field of vision on the periphery. The contact lens is given to patients one week before and again two months after immunization and is worn for five days. The contact lens is removed and horseradish peroxidase labeled antisera to each enzyme is added and incubated for ten minutes. The contacts are washed and the amount of peroxidase is measured with tetramethylbenzidine. Simple visual inspection of the spot on each contact lens with black dot developing after immunization compared to before immunization indicates the patient has mounted an immune response. These patients are expected to have complete remission eventually and long term survival. Patients whose contacts appear the same before and after immunization are expected to have disease progression and to have the same survival curves of unimmunized patients.

### EXAMPLE 3

### Detection of Liver Toxicity

The method of Example 1 is repeated with the extended wear contact lens of the same material and monoclonal antibody previously prepared to aspartate aminotransferase (SGOT) (Clin. Chim. Acta 155(3):251-62 (1986), ornithine carbamoyltransferase (Enzyme Protein 48(1) 10-17 (1994-5) and alanine aminotransferase (SGPT) (Clin. Chem. 142(3):416-9 (1996)). Each protein is independently added to separately pretreated regions on the contact lens by repeating the method of Example 1 three times on non-overlapping spots placed on the periphery of the contact lens, outside the field of vision.

The contact lens is given to normal controls and patients recently diagnosed with viral hepatitis and worn for five days. The contact lens is removed and horseradish peroxidase labeled antisera to each enzyme is added and incubated for ten minutes. The contacts are washed and the amount of peroxidase is measured with tetramethylbenzidine. Simple visual inspection of the spots on the contact lens can determine which patients have hepatitis and which are controls.

### EXAMPLE 4

### Detection of Inflammation and Organ Rejection

The method of Example 1 is repeated with the extended wear contact lens of the same material and monoclonal antibody previously prepared to serum amyloid A protein (Rinsho Byori 46(5):456-60 and 46(12):1252-7 (1998)). The spotted region was on the periphery of the contact, outside the field of vision. The contact lens is given to normal controls and patients diagnosed with active inflammation from rheumatoid arthritis and worn for five days. The contact lens is removed and horseradish peroxidase labeled rabbit antisera to serum amyloid A protein is added and incubated for ten minutes. The contacts are washed and the amount of peroxidase is measured with tetramethylbenzidine. Simple visual inspection of the spots on the contact lens can determine which patients have active rheumatoid arthritis and which are controls.

The method above will be repeated with patients who have recently received kidney transplants and the amount of serum amyloid A protein quantified in an attempt to determine which patients will undergo rejection (Nephrology Dialysis and Transplantation 10(10):1901-4 (1995),

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

We claim:

1. An ocular device comprising a transparent substrate and a plurality of specific binding molecules bound thereto, each in a discrete location, wherein said specific binding molecules selectively bind analytes contained in ocular secretions upon contacting an ocular surface with said device.

2. The device of claim 1, wherein said specific binding molecules are selected from the group consisting of nucleic acids, proteins and receptors.

3. The device of claim 2, wherein said specific binding molecules are antibodies.

4. The device of claim 1, wherein said array comprises specific binding molecules deposited at addressable locations on said device.

5. The device of claim 4, wherein said addressable locations are obtained by spotting said specific binding molecules on the surface of said device.

6. The device of claim 1, wherein said device is a contact lens.

7. The device of claim 6, wherein said contact lens is an extended wear contact lens.

8. The device of claim 4, wherein said array comprises orientation markers.

9. A method of diagnosis using ocular secretions comprising:
a) contacting an ocular device comprising a transparent substrate and a plurality of specific binding molecules bound thereto, each in a discrete location, with the ocular surface of a subject, wherein said specific binding molecules selectively bind analytes contained in ocular secretions upon contacting an ocular surface with said device;
b) allowing the ocular device and ocular fluids to interact for a specified period of time; and
c) determining the presence, absence or amount of at least one analyte present in said secretion that selectively binds to the specific binding molecules,
wherein the presence, absence or amount of said at least one analyte correlates with a specific diagnosis.

10. The method of claim 9, further comprising removing said device after said specified period of time and before said determining the presence, absence or amount of at least one analyte.

11. The method of claim 9, wherein the determining step (d) is determined by immunoassay.

12. The method of claim 9, wherein the diagnosis is determined for disease markers associated with the diseases selected from the group consisting of silent heart attacks, silent strokes, tumors and diabetes.

13. The method of claim 9, wherein the diagnosis is accomplished by specific binding molecules selective for determining levels of analyte selected from the group consisting of hormones, cytokines, lymphokines, interleukins, interferons, chemokines and tumor, viral, bacterial, fungal and T-cell antigens and nucleic acids.

14. The method of claim 9, wherein the specific binding molecules are selected from the group consisting of, nucleic acids, proteins and receptors.

15. The method of claim 14, wherein the specific binding molecules are antibodies.

16. The method of claim 9 wherein said specific binding molecules are bound to an analyte-label conjugate.

17. The method of claim 16 wherein the label is a fluorescent, quenching, optically adsorbent, reflecting or other moiety that changes the optical properties of a portion of the ocular device.

18. The method of claim 16 wherein said label is an enzyme that is inactive when bound, and upon interaction with competitive analyte in the ocular secretion, the analyte is freed thereby activating the enzyme.

19. The method of claim 18, wherein the activated enzyme metabolizes a substrate producing a color change in, on or about the device that is perceptible to said subject.

20. A method of detecting a harmful agent using a device placed on an ocular surface comprising:
(a) contacting an ocular device comprising a transparent substrate and a plurality of specific binding molecules bound thereto, each in a discrete location, with the ocular surface of a subject, wherein said specific binding molecules selectively bind analytes from the surrounding ambient environment,
(b) allowing the ocular device and ambient environment to interact for a specified period of time; and
(c) determining the presence, absence or amount of a harmful agent in said environment that selectively binds to the specific binding molecules,
wherein the presence of toxic agent binding correlates with a presence of a toxic agent in the ambient environment.

21. The method of claim 20, wherein said harmful agent is selected from the group consisting of tear gas, mustard gas, nerve gas, toxins and biologics.

22. The method of claim 20, wherein said harmful agent is generated from a high air pollution source.

23. The method of claim 22, wherein said high air pollution source is selected from the group consisting of a factory, toxic waste site, fuel station and combustion sources.

24. A method of determining the response to a vaccine comprising:
a) contacting an ocular device comprising at least one vaccine component with an ocular surface of an immunized subject;
b) allowing the ocular device and ocular fluid to interact for a specified period of time; and
c) determining the presence of an immunoglobulin contained in said secretion that selectively binds to the vaccine component attached to the ocular device,
wherein the presence of said immunoglobulin correlates with a specific response to said vaccine.

25. The method of claim 24, wherein said vaccine is a tumor antigen.

26. The method of claim 25, wherein the tumor antigen is a non-Hodgkin's Lymphoma vaccine or an epitope thereof.

27. The method of claim 24, further comprising removing said ocular device after said specified period of time and before said determining the presence of an immunoglobulin.

28. A method of determining a toxic or effective response to a chemical comprising:
a) contacting an ocular device comprising an array having a plurality of specific binding molecules with an ocular surface of a dosed subject;
b) allowing the ocular device and ocular surface to interact for a specified period of time;
c) determining the presence of an analyte comprised in said secretion that is produced in a toxic or effective response and which selectively binds to the device,
wherein the presence of said toxic substance correlates with a negative response to said drug.

29. A method of determining the response to an allergen comprising:
a) contacting an ocular device comprising an array of allergens with an ocular surface of a subject;
b) allowing the ocular device and ocular fluid to interact for a specified period of time;
c) removing said device after specified time period;
d) determining the presence, absence or amount of IgE comprised in said secretion that selectively binds to the allergens in or on the ocular device,
wherein the presence of IgE correlates with a specific response to said allergen.

30. A method of detecting an analyte in ocular secretions comprising:
contacting an ocular device comprising a transparent substrate and having an analyte adsorbing region for reversibly adsorbing the analyte
allowing the ocular device and ocular fluids to interact for a period of time sufficient for analyte to adsorb to said ocular device
removing the ocular device
removing adsorbed analyte from the ocular device; and
determining the presence, absence or amount of at least one analyte present in said secretion that bound to the ocular device
wherein the presence, absence or amount of said at least one analyte correlates with a specific diagnosis.

31. The method of claim 30, wherein the determining step (d) is determined by immunoassay.

32. The method of claim 30, wherein the diagnosis is determined for disease markers associated with the diseases selected from the group consisting of silent heart attacks, silent strokes, tumors and diabetes.

33. A therapeutic implantable device which is capable of detecting an analyte in body fluids comprising a therapeutic implantable device having a region which is a substrate having a plurality of specific binding molecules bound thereto, each in a discrete location, wherein said specific binding molecules selectively bind analytes contained in body fluids upon contact with said device, and wherein said region does not interfere with the therapeutic function of the device.

34. The device of claim 33, wherein said specific binding molecules are selected from the group consisting of nucleic acids, proteins and receptors.

35. The device of claim 34, wherein said specific binding molecules are antibodies.

36. The device of claim 33, wherein said specific binding molecules are deposited at addressable locations on said device.

37. The device of claim 36, wherein said addressable locations are obtained by spotting said specific binding molecules on the surface of said device.

38. A vessel or conduit for holding body fluids which is capable of detecting an analyte in body fluids having a region contacting said body fluids which is a substrate having a plurality of specific binding molecules bound thereto, each in a discrete location, wherein said specific binding molecules selectively bind analytes contained in said body fluids upon contact with said vessel or conduit.

39. The device of claim 38, wherein said specific binding molecules are selected from the group consisting of nucleic acids, proteins and receptors.

40. The device of claim 39, wherein said specific binding molecules are antibodies.

41. The device of claim 38, wherein said specific binding molecules are deposited at addressable locations on said device.

42. The device of claim 41, wherein said addressable locations are obtained by spotting said specific binding molecules on the surface of said device.
